(19) United States
(12) Patent Application Publication
(10) Pub. No.: US 2023/0329647 A1
(43) Pub. Date: Oct. 19, 2023

(54) METHODS AND APPARATUS FOR DISPLAYING GLUCOSE DATA

(71) Applicant: Eli Lilly and Company, Indianapolis, IN (US)

(72) Inventors: Richard Anthony ARMADA, Westfield, IN (US); Nathan Louis BILANCIO, Chicago, IL (US); Kenneth Charles SPRY, Jr., Indianapolis, IN (US)

(21) Appl. No.: 18/041,898

(22) PCT Filed: Aug. 25, 2021
(86) PCT No.: PCT/US2021/047520

§ 371 Date: Feb. 16, 2023

Related U.S. Application Data
(60) Provisional application No. 63/074,032, filed on Sep. 3, 2020.

(51) Int. Cl. A61B 5/00; G06F 3/0485; G06F 3/04845; G06F 3/0488

(52) U.S. Cl. CPC A61B 5/7435; A61B 5/743; G06F 3/0485; G06F 3/04845; G06F 3/0488

(57) ABSTRACT

The techniques described herein relate to computerized methods and apparatus for displaying glucose data. A computing device accesses glucose data of a patient recorded over time. The computing device displays a user interface that includes a first panel with a first graph that plots the glucose data over a first time period. The first panel includes a first visual indicator associated with a subset of the glucose data plotted in the first graph. The subset of the glucose data spans a second time period that is shorter in duration than the first time period. The user interface also includes a second panel with a second graph that plots the subset of the glucose data over the second time period. The second panel includes a second visual indicator associated with one or more individual glucose measurements in the second graph. The second panel includes a third panel displaying each of the one or more individual glucose measurements in numerical format.

### BACKGROUND

The pancreas regulates a person's glucose levels, but people with diabetes typically have a diminished ability to regulate their own glucose levels. If glucose levels drop too low, patients can enter a dangerous condition called hypoglycemia. If their glucose levels go too high, patients can enter another dangerous condition called hyperglycemia. Therefore, people with diabetes need to keep their glucose levels within a target ideal range by dosing themselves with insulin (which lowers glucose levels) or by ingesting carbohydrates and/or dosing themselves with glucagon (which raises glucose levels). Insulin can be administered in various forms, including through injections and/or using a pump. For example, insulin can be administered as a discrete dose that is injected all at once (e.g., a long-acting basal dose, or a bolus dose), or through a steady trickle that is infused using a pump over a period of multiple minutes or hours. Too much insulin can decrease glucose levels too much, sending patients into hypoglycemia. Too little insulin can leave glucose levels too high, sending patients into hyperglycemia. Therefore, diabetic patients have to dose themselves with the right amount of insulin, and at the right time.

### SUMMARY

The present disclosure relates to techniques for displaying a diabetic patient's recorded glucose data. In particular, the present disclosure relates to providing computerized user interfaces for visualizing recorded glucose information in a manner that allows a user to easily select and view glucose data when using a small display, such as a small touchscreen display (e.g., when using a mobile application running on a smartphone with a small display).

In one embodiment, the techniques provide a method for displaying glucose data using a computing device. The method includes accessing, by the computing device, glucose data of a patient recorded over time. The method includes displaying, by the computing device, a user interface including a first panel comprising a first graph that plots the glucose data over a first time period, wherein the first panel comprises a first visual indicator associated with a subset of the glucose data plotted in the first graph, and the subset of the glucose data spans a second time period that is shorter in duration than the first time period. The user interface includes a second panel comprising a second graph that plots the subset of the glucose data over the second time period, wherein the second panel comprises a second visual indicator associated with one or more individual glucose measurements in the second graph. The user interface includes a third panel displaying each of the one or more individual glucose measurement in numerical format.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a diagram of an exemplary user interface showing a homepage of daily activities of a diabetic patient, according to some embodiments.

FIG. 2 is a diagram of an exemplary user interface showing a history/logbook view of activities of a diabetic patient, according to some embodiments.

FIG. 3 is a diagram of an exemplary user interface displaying an initial historical view of glucose data of a patient, according to some embodiments.

FIG. 4 shows the exemplary user interface from FIG. 3 with the visual indicator at a second position in the first panel, according to some embodiments.

FIG. 5 shows the exemplary user interface from FIG. 3 with the visual indicator at a third position in the first panel, according to some embodiments.

FIG. 6 shows the exemplary user interface from FIG. 3 with an enlarged fourth panel showing events of interest, according to some embodiments.

FIG. 7 is a flow chart showing an exemplary computerized method for displaying glucose data, according to some embodiments.

FIG. 8 shows an illustrative implementation of a computer system that may be used to perform any of the aspects of the embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

The present disclosure relates to computer-implemented techniques for visualizing glucose data recorded from patients with diabetes. The inventors have discovered and appreciated that large amounts of glucose data can be recorded for diabetic patients over time. For example, conventional glucose measurement techniques (e.g., continuous glucose monitors (CGMs) or flash glucose monitors (FGMs)) can measure glucose values once every few minutes during 24 hour periods, accumulating hundreds or thousands of measurements over time (e.g., per day or week). Therefore, it can be cumbersome to view such large amounts of data. It can also be hard to identify important events and/or trends in such large amounts of data. This is especially the case when such large amounts of data are being viewed through a relatively small screen, such as a screen on a mobile device like a smartphone. For example, when viewing the data, a user often desires to easily and/or quickly zero-in on specific periods of interest (e.g., a 2.5 hour window of data) during that 24 hour period. When viewing that specific period of interest, a user may further desire to view a specific measurement within that window (e.g., a peak glucose reading, a trough glucose reading, etc.). However, when viewing such large amounts of data on a small screen (e.g., on a smartphone screen), the user may have difficulty navigating to the specific period of interest and/or the specific measurement of interest. Such differences can be due to inherent limitations in precisely selecting small points that are displayed along with hundreds or thousands of points on a small screen/display.

The techniques described herein provide for computer-implemented techniques (e.g., computer applications, such as mobile phone applications) that provide a user interface that includes a plurality of sections (e.g., panels) that display different granularities of recorded glucose data. For example, one panel of the user interface can include a graph that displays glucose data recorded over a first time period (e.g., a 24 hour time period), and a second panel can display a graph of a portion of that same data over a second, smaller time period (e.g., a 2.5 hour period). The first panel can include a visual indicator to indicate what portion of the data in the first panel is graphed in the second panel. A user can navigate the displayed data using the first panel and/or the second panel. For example, the user can scroll the data in the second panel for finer control to quickly make a selection (e.g., to select a measurement in the second panel to view further information on) without unwanted jitteriness that could otherwise occur if trying to make the selection using the first panel. As another example, the user can move the visual indicator on the first panel to change which data is displayed in the second panel. This can, for example, allow the user to view a zoomed-in portion of the data and to scroll through the data in an easier manner than otherwise provided by the first panel (which displays much more data than the second panel).

According to some embodiments, the user interface includes a second visual indicator (e.g., a vertical cursor) in the second panel that allows a user to select one or more measurements of interest, and a third panel to display detailed data for the selected measurement(s). Thus, according to some embodiments, in addition to displaying time-series data using two timescales simultaneously (a larger time scale in the first panel and a finer time scale in the second panel), the second panel also includes a second visual indicator that allows a user to precisely select and view (on a third panel) data for a single data point in the second panel. In particular, the second visual indicator allows users to more easily select from among the glucose data in the second panel, which has a zoomed-in view of the data compared to the first panel.

According to some embodiments, the user interface includes a fourth panel that displays events of interest that occurred during the time period associated with the first panel (e.g. insulin dosage events). The techniques can allow a user to select an event of interest in the fourth panel, and update the data graphed in the first and/or second panels. For example, upon selection of an event in the fourth panel, the computing device can move the first indicator to center around the time associated with the selected event. The computing device can additionally, or alternatively, update the data in the second panel such that the data graphed in the second panel is centered around the time of the selected event. Therefore, the techniques can allow a user to select a single event and to view glucose measurements that occurred around the same time as the event.

According to some embodiments, the computing device can automatically adjust the scale of the data graphed in the first and/or second panel to provide a consistent view of the glucose measurements. For example, depending on the frequency at which the glucose data is recorded, the computing device can automatically scale the second time period over which data is graphed for the second panel to keep measurements visually spaced about the same distance (e.g., based on number of pixels and/or a distance) regardless of the frequency at which the glucose data was recorded. As a result, regardless of the glucose measurement frequency, the user interface can provide a consistent user interface to the user.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

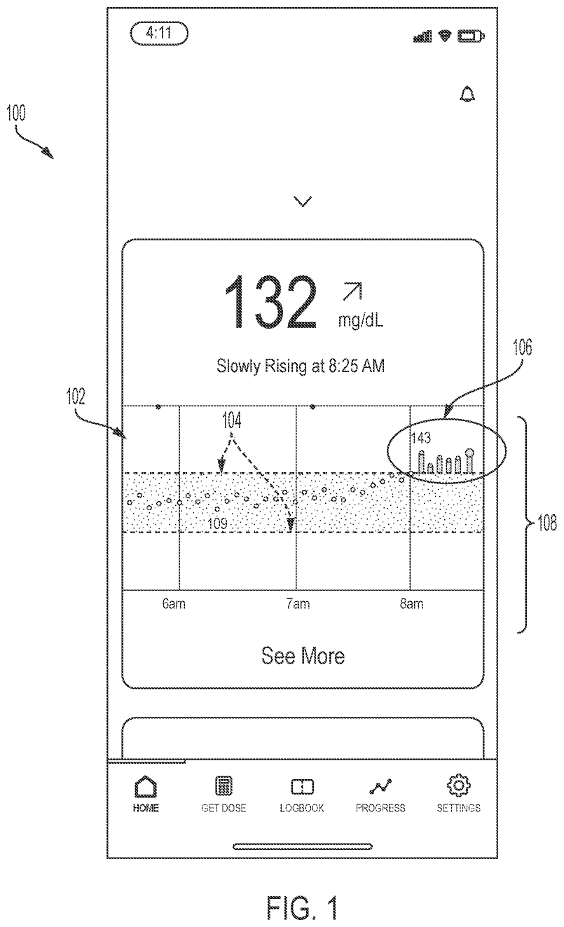

FIGS. 1-6 show a series of exemplary screenshots of the user interface presented on the display of the computing device (e.g., a smartphone running a mobile application, in this example), according to some embodiments. FIG. 1 is a diagram of an exemplary user interface 100 showing a homepage of daily activities for a diabetic patient, according to some embodiments. The user interface 100 can be used to expose daily activities in a condensed format that allows users to perceive trends, streaks, gaps in behavior, and/or the like at a glance. The user interface 100 includes a graph 108 that graphs glucose measurements that occurred between approximately 5:30 am to 8:30 am on a particular day, and indicates that at 8:25 AM the diabetic patient has a slowly rising glucose value of 132 mg/dL. According to some embodiments, in order to emphasize high and low value's distances from the normal range, high and/or low values can be called out by both a color and/or visually by grounding the bar to the normal range. In this example, the user interface 100 includes a graph 102 with visual indicators 104 to indicate a normal range (e.g., in this example, the visual indicators specify a horizontal area), and the portion 106 includes bars grounding the measurements in the portion 106 to the horizontal line 104.

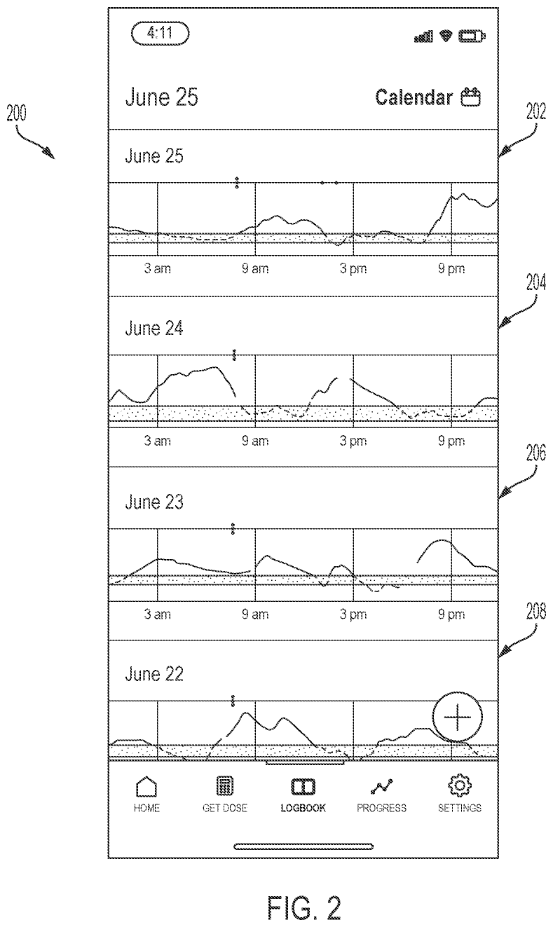

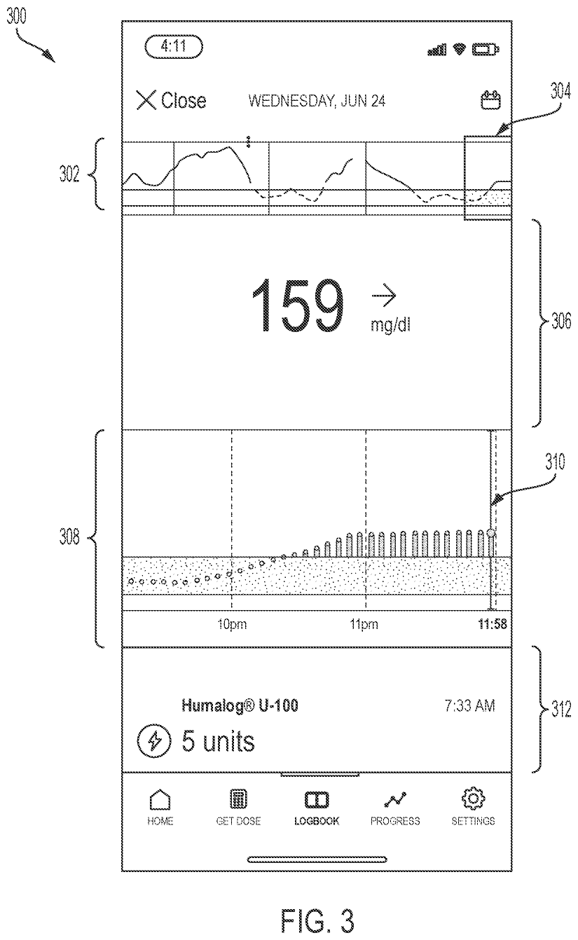

FIG. 2 is a diagram of an exemplary user interface 200 showing a history/logbook view for a diabetic patient, according to some embodiments. The user interface 200 exposes the activities (glucose measurements) over a series of time periods (e.g., over a series of days 202-208 which, in this example, correspond to the days June 22 through June 25) in a condensed format that allows users to find trends, streaks, gaps in behavior, and/or the like, at a glance. In this example, each day 202-208 includes a line graph that plots the glucose values for that particular day chronologically from left to right. As described further herein, the user interface 200 provides the daily activities in a format similar to that used in other user interfaces (e.g., first panel 302 in FIG. 3) that allow the user to inspect sets of data and/or individual measurement(s). Therefore, the user interface 200 includes entries that prime users for how they can inspect the data according to the techniques described herein. According to some embodiments, a user can select the line graph for a particular day to open one or more user interfaces to view further details for the particular selected day. For example, selecting the line graph for a particular day can open the exemplary user interface 300 of FIG. 3.

FIG. 3 is a diagram of an exemplary user interface 300 displaying an initial historical view of glucose data of a patient, according to some embodiments. The user interface 300 has a first panel 302, which in this example is at the top of the user interface 300. While the description herein refers to various panels in the user interface, including the first panel 302, it should be appreciated that this is for illustrative purposes and is used to refer to a respective portion of the user interface. The first panel 302 includes a graph that plots the recorded glucose values recorded over a time period (e.g., a 24 hour period). In this example, the graph is a line graph that plots the glucose values chronologically from left to right. According to some embodiments, the first panel 302 can display the selected day from the user interface 200 in FIG. 2. The first panel 302 includes a first visual indicator, in this example a rectangular box 304. The user of the device can drag the visual indicator box 304 left and right along the timeline of the first panel 302 (e.g., via a touchscreen and/or other input device, such as a computer mouse).

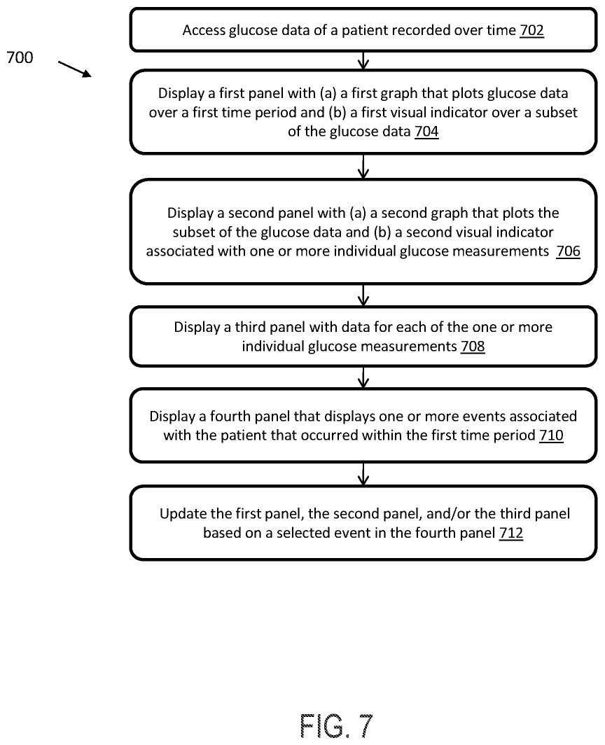

The user interface 300 also has a second panel 308 that includes a graph that displays a close-up view of the glucose values within the rectangular box 304. The second panel is towards the middle of the user interface 300, below the fourth panel 306 and above the third panel 312. Thus, compared to the first panel 302, the second panel 308 displays glucose values over a shorter time period (e.g., 2.5 hours as opposed to 24 hours) within the time period displayed in the first panel 302. The second panel 308 also includes a second visual indicator 310, which in this example is a vertical cursor that is positioned towards the right of second panel 308. As described in conjunction with FIG. 7, according to some embodiments, as the user slides the first visual indicator 304 along the plotted data in the graph of the first panel 302, the data graphed in the second panel changes accordingly. According to some embodiments, as the user slides the graph in the second panel 308 (e.g., by touching or clicking anywhere in the area of second panel 308 and dragging the touch- or click-point to the left or right), the visual indicator 304 in the first panel 302 moves to be in association with the data displayed in the second panel 308 (e.g., as also described further in conjunction with FIG. 7).

According to some embodiments, the user interface 300 also includes a third panel 306 that displays data associated with the measurement(s) associated with the second visual indicator 310. In this example, the third panel 306 displays the measurement displayed in the second panel 308 with which the vertical cursor 310 is aligned (in this example, 159 mg/dL). The third panel 306 can therefore provide a display that allows a user to easily view specific glucose measurement data for the measurement(s) selected in the second panel 308 using the second visual indicator.

According to some embodiments, the user-interface can also include a fourth panel 312. That fourth panel displays events of interest that happened during the time period covered by the first panel 304 (e.g., events that occurred during the 24 hour period of glucose measurements). At least some of the events displayed may be events that affect, or are expected to affect, the glucose levels of the diabetic patient. The events of interest can include, for example, doses of meal-time insulin taken (in this example, a dose of Humalog U-100, 5 units), doses of basal or long-acting insulin taken (e.g., Basaglar), amounts of carbs ingested, and/or the like. Each event of interest can include a time stamp indicating when that event occurred (e.g., day and/or time) and indicate how the event was logged. For example, the event can be automatically logged by a connected delivery device (e.g., by an insulin injection device) or manually logged (e.g., by the person with diabetes). Although only one event is shown in FIG. 3, in some embodiments the panel 312 can display multiple events and/or be controlled (e.g., scrolled up or down) to see more events. According to some embodiments, when a particular event is selected in the panel 312, the computing device can update one or more of the first panel 302 and/or second panel 308 accordingly. For example, upon selection of a particular event in the panel 312, the computing device can automatically scroll the first visual indicator 304 and the second panel 308 over to center on the glucose data around the time period of the event selected in the fourth panel 312 (e.g., allowing the user to see glucose values just before and after that event of interest). In some embodiments, the glucose data displayed by the computing device after selection of a particular event may not be precisely centred on the time period of the event, but may be offset in time to centre on a time period slightly before or slightly after the selected event.

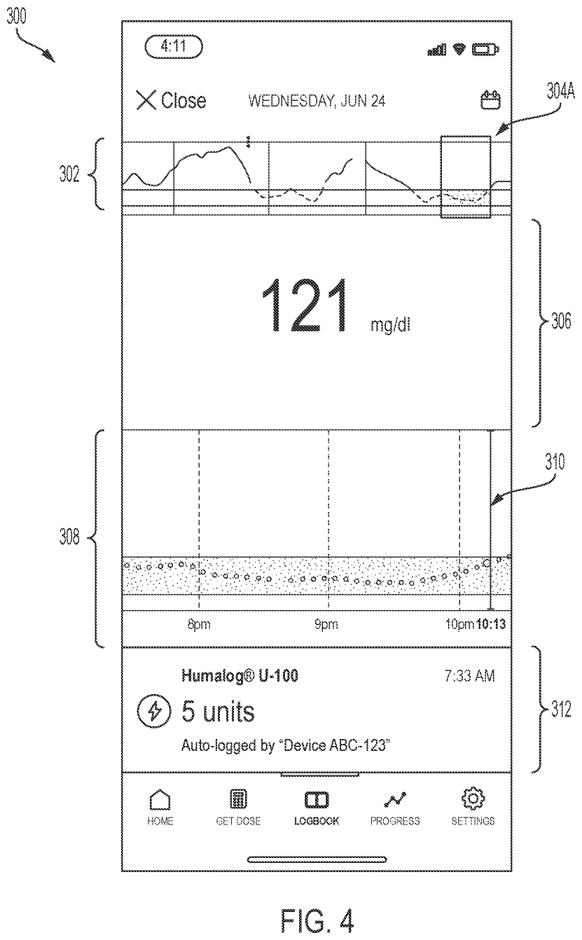

FIG. 4 shows the exemplary user interface 300 from FIG. 3 with the visual indicator 304 at a second position (shown as 304A) in the first panel 302, according to some embodiments. As explained herein, the data graphed in the second panel 308 is based on the position of the visual indicator 304A in the first panel 302, such that the second panel 308 graphs a close-up view of the glucose values in the first panel 302 within the rectangular box 304A. Similarly, the third panel 306 displays the measurement with which the vertical cursor 310 is aligned in the second panel 308 (in this example, 121 mg/dL). As described in conjunction with FIG. 7, the visual indicator 304A can be moved to the second position by scrolling the graph in the second panel 308, by moving the visual indicator 304, and/or the like.

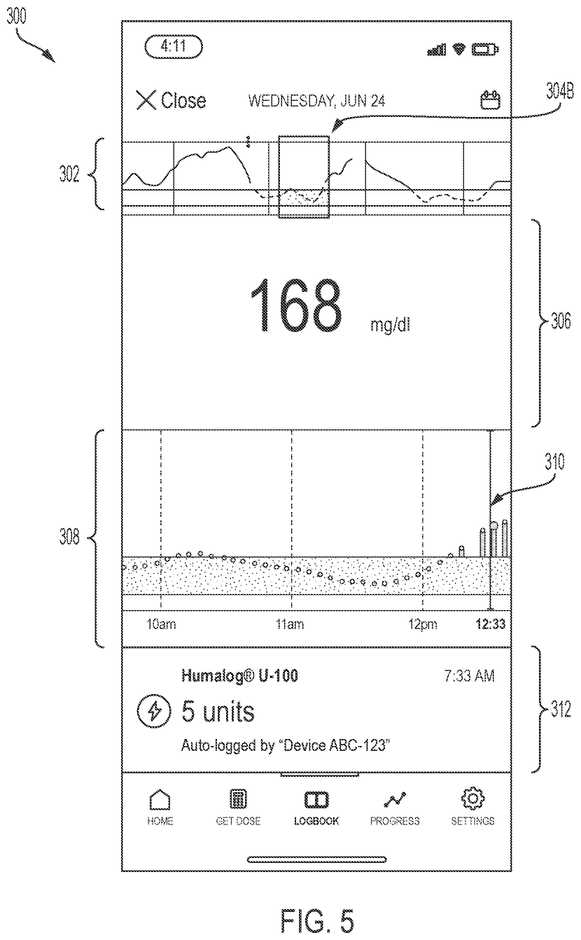

FIG. 5 shows the exemplary user interface 300 from FIG. 3 with the visual indicator 304 at a third position (shown as 304B) in the first panel 302, according to some embodiments. The second panel 308 graphs a close-up view of the glucose values in the first panel 302 within the rectangular box 304B. The third panel 306 displays the measurement with which the vertical cursor 310 is aligned in the second panel 308 (in this example, 168 mg/dL).

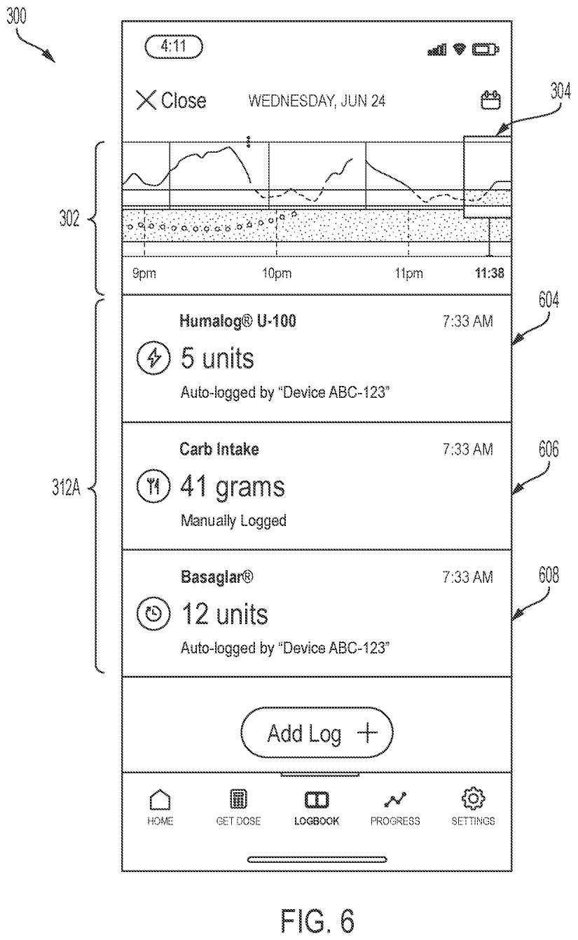

FIG. 6 shows the exemplary user interface 300 from FIG. 3 with an enlarged fourth panel 312 (shown as 312A), according to some embodiments. The fourth panel 312 can be enlarged, for example, by tapping on the fourth panel 312, scrolling the fourth panel 312, and/or otherwise causing the fourth panel 312 to enlarge. The enlarged fourth panel 312 in this example displays event details for three events that occurred on the selected day. The first event 604 indicates that 5 units of Humalog U-100 was auto-logged by Device ABC-123 (e.g., a smart pen, a smart device attachable to an auto injector, and/or the like) at 7:33 AM. The second event 606 indicates that 41 grams of carb intake were manually logged at 7:33 AM. The third event 608 indicates that 12 units of Basaglar was auto-logged by Device ABC-123 at 7:33 AM. As shown, the events are recorded for 7:33 AM, and therefore are independent of the location of the visual indicator 304 in the first panel 302, which is associated with events that occurred after 11 pm that day.

FIG. 7 is a flow chart showing an exemplary computerized method for displaying glucose data, according to some embodiments. At step 702, the device (e.g., a smartphone) accesses glucose data of a patient recorded over time. At step 704, the device displays a user interface that includes a plurality of panels. The user interface includes a first panel (e.g., at a top of the screen, such as the first panel 302 in FIG. 3) that includes a first graph that plots the glucose data over a first time period (e.g., glucose data recorded over a 12 or 24 hour period). The first panel also displays a first visual indicator associated with a subset of the glucose data plotted in the first graph that spans a second time period (e.g., 1 hour, 2.5 hours, etc.) that is shorter in duration than the first time period. The first visual indicator can be, for example, a rectangular box displayed over/around the subset of the glucose data, such as shown as 304 in FIG. 3. At step 706, the device displays a second panel (e.g., panel 308 in FIG. 3) that includes a second graph that plots the subset of the glucose data over the second time period. The second panel also displays a second visual indicator (e.g., a vertical cursor, such as shown as 310 in FIG. 3) that is associated with one or more individual glucose measurements in the second graph. At step 708, the device displays a third panel (e.g., third panel 306 in FIG. 3) that displays data (e.g., numerical data) associated with each of the one or more individual glucose measurements. At step 710, the device displays a fourth panel that displays one or more events (e.g., insulin dose(s), ingested carbs, etc.) associated with the patient that occurred within the first time period. At step 712, the device updates the first panel, the second panel, and/or the third panel based on a selected event in the fourth panel.

Referring to step 702, the glucose data can be recorded manually and/or automatically. In some embodiments, the user is connected to and/or views data collected by monitoring devices that provide input data to the computing device. For example, a patient (e.g., the user) can be connected to a CGM or FGM that provides the user's glucose reading to the computing device. In some embodiments, the glucose data can be provided by a Blood Glucose Monitor (BGM) that either automatically provides the glucose reading to the computing device, or that is read by the user and manually input into the computing device. According to some embodiments, some and/or all of the glucose data is stored on the computing device. According to some embodiments, some and/or all of the glucose data is stored on a separate computing device, such as on a server, a cloud computing platform, the CGM, the FGM, the BGM, and/or the like.

Referring to step 704, the first indicator can comprise any shape suitable for indicating a desired portion of glucose data in the first panel to view (e.g., in a finer-grained view in a second panel). According to some embodiments, the first visual indicator can be a rectangle displayed around a subset of the glucose data plotted in the first graph, such as shown in FIGS. 3-6. According to some embodiments, the computing device can receive input data indicative of a user of the computing device moving the first visual indicator from a current position in the first panel to a new position in the first panel (e.g., by touching and dragging the visual indicator left and right along the timeline of the first panel). The computing device can update the first panel to display the first visual indicator at the new position, such that the first visual indicator is associated with a second subset of the glucose data at the new position that is at least partially different than the subset of the glucose data.

According to some embodiments, as the user moves the visual indicator, the glucose measurements graphed in the second panel can be updated (e.g., scrolled) to reflect the values associated with the visual indicator. For example, the computing device can update the second panel to update the second graph to plot the second subset of the glucose data. For example, referring to FIG. 3, the user can move the visual indicator 304 left and right along the timeline of the first panel 302, and the values graphed in the second panel 308 also scroll left and right correspondingly. As shown in FIG. 4, the visual indicator 304 is at a second position 304A, and the values in the second panel 308 are those associated with the second position 304A. Similarly, the visual indicator 304 is at a third position 304B in FIG. 5, and the values in the second panel 308 are those associated with the third position 304B. By moving the first visual indicator in the first panel 302 (e.g., by dragging the first visual indicator left and right), the user interface provides the user with coarse control over what glucose values to display in the second panel 308.

Referring to step 706, according to some embodiments the second visual indicator can be associated with a single glucose measurement plotted in the second graph. For example, the second visual indicator can be a vertical bar displayed at the single glucose measurement. In other examples, the second visual indicator can include one or more arrows, lines, and/or symbols. In some embodiments, the second visual indicator can be associated with more than one glucose measurement, and may instead be associated with two, three, four, or more glucose measurements. According to some embodiments, the computing device receives input data indicative of a user of the computing device moving the second graph in a direction (e.g., sliding the second graph left or right). The computing device can update the second panel based on the received input to update the second graph to plot a new subset of the glucose data that is at least partially different than the subset of the glucose data. For example, the computing device can shift the data plotted in the second graph to plot glucose measurements earlier or later in time, effectively shifting the graph left or right to plot data to the left or right of the current data, respectively.

According to some embodiments, the computing device can update the first panel to display the first visual indicator at a new position associated with the new subset of the glucose data graphed in the second graph. For example, referring to FIG. 3, when the user places his/her finger anywhere on the second panel 308 and moves his/her finger left or right on the screen, the entire graph of glucose data in the second panel 308 shifts to graph glucose data to the left or right, accordingly. The rectangular first visual indicator 304 on first panel 302 also moves left and/or right, accordingly. By dragging the graph in the second panel 308 left and right, the user has fine control over what glucose values are displayed in the second panel 308. In some embodiments, the second visual indicator does not move as the user drags the graph in the second panel 308 left and right. When the user releases the second panel 308, whichever glucose measurement is aligned with the second visual indicator becomes associated with the second visual indicator. If there is no single glucose measurement that is precisely aligned with the second visual indicator when the user releases the second panel 308, then in some embodiments, the computing device can automatically move the second panel 308 slightly such that the single glucose measurement that is closest to the second visual indicator is “snapped” to the second visual indicator. This user-interface is particularly advantageous for selecting a single glucose measurement on a small screen, such as a smartphone screen. By allowing the user to touch and manipulate the entire area of second panel 308 rather than the relatively small second visual indicator, the user is afforded greater precision and control over which single glucose measurement is selected by the second visual indicator.

Referring to step 708, the third panel displays glucose measurements in numerical format (e.g., in mg/dL) for each of the one or more glucose measurements associated with the second visual indicator. For example, as shown in FIG. 3, the third panel 306 displays 159 mg/dL for the glucose measurement associated with the second visual indicator 310 in the second panel 308. If the second visual indicator is associated with more than one glucose measurement, the third panel 306 can display a different number (e.g., in mg/dL) for each associated glucose measurement.

Referring to step 710, as described herein, according to some embodiments the user interface includes a fourth panel that displays (e.g., via a scrollable display) one or more events associated with the patient that occurred within the first time period. The one or more events can include an administered dose of meal-time insulin, an administered dose of basal insulin, an administered dose of long-acting insulin, an amount of ingested carbs, or some combination thereof. The one or more events can be related to the glucose measurements based on the time recorded, but otherwise can be different from the actual glucose measurements.

Referring to step 712, according to some embodiments, the computing device may update the first panel, the second panel, and/or the third panel based on a selected event in the fourth panel. For example, the computing device can receive input data indicative of a user of the computing device selecting an event in the fourth panel. The computing device can update the second panel to display a second subset of the glucose data centered around (or at least include, if not precisely centered around) a time period associated with the selected event, wherein the second subset of the glucose data is at least partially different than the subset of the glucose data. Additionally, or alternatively, the computing device can update the first panel to display the first visual indicator so that the first visual indicator is associated with the second subset of the glucose data.

According to some embodiments, the computing device can adapt one or more aspects of the user interface based on the measurement frequency of the glucose measurement device (e.g., CGM or FGM). For example, some glucose measurement devices may take measurements every 3 minutes, while others may measure every 5 minutes or 10 minutes. As another example, some glucose measurement devices can be configured to measure at different frequencies. According to some embodiments, the computing device can be configured to dynamically adjust aspects of the user interface to provide a consistent presentation of the glucose data (e.g., even if the data has different characteristics, such as data frequency, etc.). As an illustrative example, the computing device can configure the user interface to display glucose data that was recorded at a first frequency. For example, the computing device can determine a first range of data associated with the first visual indicator based on the first frequency, which in turn can control the number of glucose measurements displayed in the second panel. The computing device can then access second glucose data recorded at a second (different) frequency, and determine, based on the second frequency, a second (different) range of data associated with the first visual indicator. Thus, the range of data, such as the time period covered by the first visual indicator, can be larger and/or smaller to capture approximately a similar number of glucose measurements. As a result, the second panel can display a similar number of glucose measurements for the data recorded at both the first frequency and the second frequency. Referring to FIG. 3, for example, the user-interface 300 can be configured to automatically adjust the time-scale of the second panel 308 (and correspondingly, the width of the box of the visual indicator 304) so that the visual distance between two consecutive glucose device measurements remains the same on the screen (e.g., 5 mm apart, 50 pixels apart, and/or the like), regardless of the data frequency. Thus, in some embodiments, instead of hard coding the time scale of the second panel 308 to always show a certain time period (e.g., 2.5 hours), the computing device can automatically vary the time scale of the second panel based on the frequency of the glucose device's measurements.

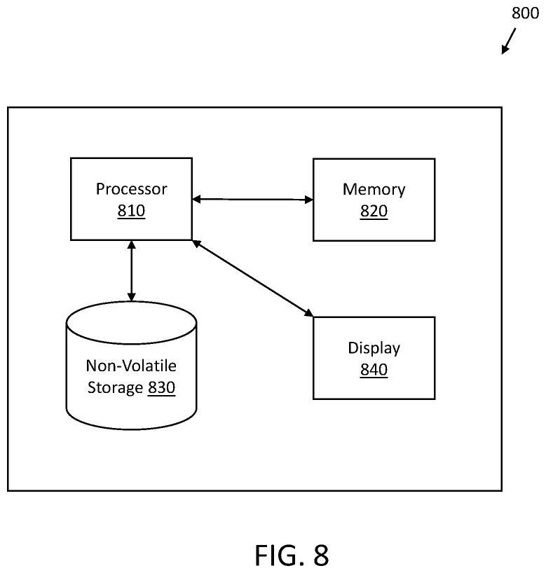

An illustrative implementation of a computer system 800 that may be used to perform any of the aspects of the techniques and embodiments disclosed herein is shown in FIG. 8. The computer system 800 may include one or more processors 810 and one or more non-transitory computer-readable storage media (e.g., memory 820 and one or more non-volatile storage media 830) and a display 840. The processor 810 may control writing data to and reading data from the memory 820 and the non-volatile storage device 830 in any suitable manner, as the aspects of the invention described herein are not limited in this respect. To perform functionality and/or techniques described herein, the processor 810 may execute one or more instructions stored in one or more computer-readable storage media (e.g., the memory 820, storage media, etc.), which may serve as non-transitory computer-readable storage media storing instructions for execution by the processor 810.

In connection with techniques described herein, code used to, for example, provide user interfaces to visualize glucose measurements and related information may be stored on one or more computer-readable storage media of computer system 800. Processor 810 may execute any such code to provide any techniques for displaying glucose data, as described herein. Any other software, programs or instructions described herein may also be stored and executed by computer system 800. It will be appreciated that computer code may be applied to any aspects of methods and techniques described herein. For example, computer code may be applied to interact with an operating system to generate the user interfaces described herein through conventional operating system processes.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This allows elements to optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting.

Various aspects are described in this disclosure, which include, but are not limited to, the following aspects:
(1) A method for displaying glucose data using a computing device, the method comprising: accessing, by the computing device, glucose data of a patient recorded over time; displaying, by the computing device, a user interface comprising: a first panel comprising a first graph that plots the glucose data over a first time period, wherein: the first panel comprises a first visual indicator associated with a subset of the glucose data plotted in the first graph, and the subset of the glucose data spans a second time period that is shorter in duration than the first time period; a second panel comprising a second graph that plots the subset of the glucose data over the second time period, wherein the second panel comprises a second visual indicator associated with one or more individual glucose measurements in the second graph; and a third panel displaying each of the one or more individual glucose measurement in numerical format.
(2) The method of aspect 1, further comprising: receiving input data indicative of a user of the computing device moving the first visual indicator from a current position in the first panel to a new position in the first panel; and updating the first panel to display the first visual indicator at the new position, wherein the first visual indicator is associated with a second subset of the glucose data at the new position that is at least partially different than the subset of the glucose data.
(3) The method of aspect 2, further comprising: updating the second panel to update the second graph to plot the second subset of the glucose data.
(4) The method of aspect 1, further comprising: receiving input data indicative of a user of the computing device sliding the second graph in a direction; and updating, based on the received input, the second panel to update the second graph to plot a new subset of the glucose data that is at least partially different than the subset of the glucose data.
(5) The method of aspect 4, further comprising: updating the first panel to display the first visual indicator at a new position associated with the new subset of the glucose data.
(6) The method of any of aspects 1-5, wherein the first visual indicator comprises a rectangle displayed around the subset of the glucose data plotted in the first graph.
(7) The method of any of aspects 1-6, wherein the second visual indicator is associated with a single glucose measurement plotted in the second graph.
(8) The method of aspect 7, wherein the second visual indicator comprises a vertical bar displayed at the single glucose measurement.
(9) The method of any of aspects 1-8, wherein the glucose data is recorded, at least in part, by a continuous glucose monitor (CGM) attached to the patient.
(10) The method of any of aspects 1-9, wherein: the accessed glucose data was recorded at a first frequency; and the first visual indicator comprises a first range determined based on the first frequency to capture the subset of the glucose data; and the method further comprising: accessing second glucose data recorded at a second frequency; and determining, based on the second frequency, a second range for the first visual indicator that is different than the first range.
(11) The method of any of aspects 1-10, wherein displaying the user interface further comprises displaying a fourth panel comprising a scrollable display of one or more events associated with the patient that occurred within the first time period.
(12) The method of aspect 11, wherein the one or more events comprises an administered dose of meal-time insulin, an administered dose of basal insulin, an administered dose of long-acting insulin, an amount of ingested carbs, or some combination thereof.
(13) The method of aspect 11, further comprising: receiving input data indicative of a user of the computing device selecting an event in the fourth panel; updating the second panel to display a second subset of the glucose data measured during a time period associated with the selected event, wherein the second subset of the glucose data is at least partially different than the subset of the glucose data; and updating the first panel to display the first visual indicator so that the first visual indicator is associated with the second subset of the glucose data.
(14) A non-transitory computer-readable media comprising instructions that, when executed by one or more processors on a computing device, are operable to cause the one or more processors to execute the method of any of aspects 1-13.
(15) A system comprising a memory storing instructions, and a processor configured to execute the instructions to perform the method of any of aspects 1-13.

1. A method for displaying glucose data using a computing device, the method comprising:
accessing, by the computing device, glucose data of a patient recorded over time; and
displaying, by the computing device, a user interface comprising:
a first panel comprising a first graph that plots the glucose data over a first time period, wherein:
the first panel comprises a first visual indicator associated with a subset of the glucose data plotted in the first graph, and
the subset of the glucose data spans a second time period that is shorter in duration than the first time period;
a second panel comprising a second graph that plots the subset of the glucose data over the second time period, wherein the second panel comprises a second visual indicator associated with one or more individual glucose measurements in the second graph; and
a third panel displaying each of the one or more individual glucose measurement in numerical format.

2. The method of claim 1, further comprising:
receiving input data indicative of a user of the computing device moving the first visual indicator from a current position in the first panel to a new position in the first panel; and
updating the first panel to display the first visual indicator at the new position, wherein the first visual indicator is associated with a second subset of the glucose data at the new position that is at least partially different than the subset of the glucose data.

3. The method of claim 2, further comprising:
updating the second panel to update the second graph to plot the second subset of the glucose data.

4. The method of claim 1, further comprising:
receiving input data indicative of a user of the computing device sliding the second graph in a direction; and
updating, based on the received input data, the second panel to update the second graph to plot a new subset of the glucose data that is at least partially different than the subset of the glucose data.

5. The method of claim 4, further comprising:
updating the first panel to display the first visual indicator at a new position associated with the new subset of the glucose data.

6. The method of claim 1, wherein the first visual indicator comprises a rectangle displayed around the subset of the glucose data plotted in the first graph.

7. The method of claim 1, wherein the second visual indicator is associated with a single glucose measurement plotted in the second graph.

8. The method of claim 7, wherein the second visual indicator comprises a vertical bar displayed at the single glucose measurement.

9. The method of claim 1, wherein the glucose data is recorded, at least in part, by a continuous glucose monitor (CGM) attached to the patient.

10. The method of claim 1,
wherein:
the accessed glucose data was recorded at a first frequency; and
the first visual indicator comprises a first range determined based on the first frequency to capture the subset of the glucose data; and
the method further comprising:
accessing second glucose data recorded at a second frequency; and
determining, based on the second frequency, a second range for the first visual indicator that is different than the first range.

11. The method of claim 1, wherein displaying the user interface further comprises displaying a fourth panel comprising a scrollable display of one or more events associated with the patient that occurred within the first time period.

12. The method of claim 11, wherein the one or more events comprises an administered dose of meal-time insulin, an administered dose of basal insulin, an administered dose of long-acting insulin, an amount of ingested carbs, or some combination thereof.

13. The method of claim 11, further comprising:
receiving input data indicative of a user of the computing device selecting an event in the fourth panel;
updating the second panel to display a second subset of the glucose data measured during a time period associated with the selected event, wherein the second subset of the glucose data is at least partially different than the subset of the glucose data; and
updating the first panel to display the first visual indicator so that the first visual indicator is associated with the second subset of the glucose data.

14. A non-transitory computer-readable media comprising instructions that, when executed by one or more processors on a computing device, are operable to cause the one or more processors to;
access glucose data of a patient recorded over time; and
display a user interface comprising:
a first panel comprising a first graph that plots the glucose data over a first period, wherein:
the first panel comprises a first visual indicator associated with a subset of the glucose data plotted in the first graph, and
the subset of the glucose data spans a second time period that is shorter in duration than the first time period:
a second panel comprising a second graph that plots the subset of the glucose data over the second time period, wherein the second panel comprises a second visual indicator associated with one or more individual glucose measurements in the second graph; and
a third panel displaying each of the one or more individual glucose measurements in numerical format.

15. A system comprising a memory storing instructions, and a processor configured to execute the instructions to:
access glucose data of a patient recorded over time; and
display a user interface comprising:
a first panel comprising a first graph that plots the glucose data over a first period, wherein:
the first panel comprises a first visual indicator associated with a subset of the glucose data plotted in the first graph, and
the subset of the glucose data spans a second time period that is shorter in duration than the first time period:
a second panel comprising a second graph that plots the subset of the glucose data over the second time period, wherein the second panel comprises a second visual indicator associated with one or more individual glucose measurements in the second graph; and
a third panel displaying each of the one or more individual glucose measurements in numerical format.

16. The non-transitory computer-readable media of claim 14, wherein the instructions are further operable to cause the one or more processors to:
receive input data indicative of a user of the computing device moving the first visual indicator from a current position in the first panel to a new position in the first panel; and
update the first panel to display the first visual indicator at the new position, wherein the first visual indicator is associated with a second subset of the glucose data at the new position that is at least partially different than the subset of the glucose data.

17. The non-transitory computer-readable media of claim 16, wherein the instructions are further operable to cause the one or more processors to update the second panel to update the second graph to plot the second subset of the glucose data.

18. The non-transitory computer-readable media of claim 14, wherein the instructions are further operable to cause the one or more processors to:
receive input data indicative of a user of the computing device sliding the second graph in a direction; and
update, based on the received input data, the second panel to update the second graph to plot a new subset of the glucose data that is at least partially different than the subset of the glucose data.

19. The non-transitory computer-readable media of claim 18, wherein the instructions are further operable to cause the one or more processors to update the first panel to display the first visual indicator at a new position associated with the new subset of the glucose data.

20. The non-transitory computer-readable media of claim 14, wherein displaying the user interface further comprises displaying a fourth panel comprising a scrollable display of one or more events associated with the patient that occurred within the first time period.

21. The non-transitory computer-readable media of claim 20, wherein the one or more events comprises an administered dose of meal-time insulin, an administered dose of basal insulin, an administered dose of long-acting insulin, an amount of ingested carbs, or some combination thereof.

22. The non-transitory computer-readable media of claim 21, wherein the instructions are further operable to cause the one or more processors to:
receive input data indicative of a user of the computing device selecting an event in the fourth panel;
update the second panel to display a second subset of the glucose data measured during a time period associated with the selected event, wherein the second subset of the glucose data is at least partially different than the subset of the glucose data; and
update the first panel to display the first visual indicator so that the first visual indicator is associated with the second subset of the glucose data.
